(19) United States
(12) Patent Application Publication
(10) Pub. No.: US 2025/0090361 A1
(43) Pub. Date: Mar. 20, 2025

(54) Belt with knob for adjustable elasticity

(71) Applicant: Dongguan Fushun Sports Goods Co., Ltd., Dongguan (CN)

(72) Inventor: Xiaogao Gu, Ji'an (CN)

(21) Appl. No.: 18/474,049

(22) Filed: Sep. 25, 2023

(30) Foreign Application Priority Data
Sep. 14, 2023 (CN) 2022230859194

(51) Int. Cl. A61F 5/02

(52) U.S. Cl. CPC A61F 5/028; A41F 9/00; A41F 9/02; A41F 9/025

(57) ABSTRACT

A belt with knob for adjustable elasticity is disclosed, including a belt body. The belt body includes a support part and fixing belts connected to both sides of the support part, the support part is configured to be connected with a plurality of adjusting belts, and the adjusting belts are arranged symmetrically along a center of the support part to the both sides. Two fixing belts are configured with adjusting knobs, the adjusting knobs are configured with elastic strings, and the adjusting knobs are connected with the adjusting belts of corresponding sides by linkage of the elastic strings. The belt of the present disclosure can improve effects of tightening and pulling, and thereby achieving adjusting elasticity effect. Moreover, due to the adjusting knobs are one-way adjusting knobs, which can effectively prevent human body muscles from contracting and expanding action influencing an optimal adjustment of the belt elasticity.

### TECHNICAL FIELD

The present application relates to the technical field of support belt, and specifically to a novelty of belt with knob for adjustable elasticity.

### BACKGROUND

Waist protectors, also known as beam waist or support belt. In sports, the support belt is usually tied around waist to protect the waist from injury, insulation, or some special functions, and in sports the waist is often tense and painful because of excessive resistance exercise resulting in high intensity muscle loading bearing. Wearing appropriate materials and specification of support belt may effectively protect lumbar tissues and prevent sports injuries. The support belt is generally made of breathable support belt net, high-quality elastic band, and elastic fabric, etc., where a semi-hard aluminum alloy strip or medical fiber plastic strips are hidden inside. Moreover, the support further is also configured with an excellent Velcro to support and fix spine and tight a pull force and peel strength of abdomen, to ensure that the support belt may be used safely for a long period of time, and thus facilitate adjustment of a size of the support belt and tighten the support belt to improve an abdominal pressure, reduce a lumbar spine load-bearing, limit lumbosacral spine activities, and slow down the pain. Thus such support belt has an auxiliary physical therapy rehabilitation unique design, anti-allergic, breathable, comfortable to wear, elastic free adjustment, and has a certain role in the protection of the spine.

Existing support belt is generally worn by following two ways to connect. I), through the Velcro on both sides of fixing belts to connect, but this way does not be adjusted through the elastic band to achieve a best support effect. II), through adjusting buckles to connect, this way may be through the fixing belts in adjusting buckles back and forth sliding to achieve an effect of adjusting elasticity, but human muscle will undergo a contraction and expansion action when the human body is in motion, which will cause the fixing belts to slide within the adjusting buckles, affecting an optimally adjusted belt elasticity and leading to poor protective care.

### SUMMARY

The present disclosure provides a novelty of belt with knob for adjustable elasticity to address above problems mentioned in the background.

To achieve above objectives, the present disclosure proposes following technical solutions. In some embodiments of the present disclosure, a novelty of belt with knob for adjustable elasticity is provided, including a belt body. The belt body includes a support part and fixing belts connected to both sides of the support part, the support part is configured to be connected with a plurality of adjusting belts, and the adjusting belts are arranged symmetrically along a center of the support part to the both sides. Two fixing belts are configured with adjusting knobs, the adjusting knobs are configured with elastic strings, and the adjusting knobs are connected with the adjusting belts of corresponding sides by linkage of the elastic strings.

In some preferred embodiments of the present disclosure, the he adjusting knobs are one-way adjusting knobs.

In some preferred embodiments of the present disclosure, the support part is configured with a plurality of evenly spaced support strips inside.

In some preferred embodiments of the present disclosure, either of the fixing belts is configured with Velcro, and the two fixing belts are configured with loops adapted to the Velcro.

In some preferred embodiments of the present disclosure, the fixing belts also have gauzes sewn to them, the gauzes are located above the elastic strings, and there are openings between the gauzes and the fixing belts for the elastic strings to pass through.

In some preferred embodiments of the present disclosure, a plurality of air holes is uniformly provided on an inner side of the fixing belts.

Compared with existing technologies, the present disclosure at least has following advantages.

By setting the adjusting knobs and the adjusting belts, the two fixing belts surround user's waist and are fixedly connected, and then by rotating the adjusting knobs, the adjusting belts are pulled to both sides by the elastic strings to improve tightening and pulling effects, which makes the belt of the present disclosure can achieve an effect of adjusting tightness. Moreover, the adjusting knobs are designed as one-way adjusting knobs, which can effectively prevent human body muscles from actions of contracting and expanding to affect a best adjusting elasticity of the belt.

### BRIEF DESCRIPTION OF THE DRAWINGS

FIG. 1 shows a schematic diagram of a belt in accordance with some embodiments of the present disclosure.

FIG. 2 shows another schematic diagram of a belt in accordance with some embodiments of the present disclosure.

FIG. 3 shows still another schematic diagram of a belt in accordance with some embodiments of the present disclosure.

### DETAILED DESCRIPTION OF THE EMBODIMENTS

The technical solutions in the embodiments of the present disclosure will be described clearly and completely in the following in conjunction with the accompanying drawings, and it is obvious that the described embodiments are only a part of embodiments of the present disclosure and not all of the embodiments. Based on the embodiments of the present disclosure, all other embodiments obtained by a person of ordinary skill in the art without creative labor are within the scope of protection of the present disclosure.

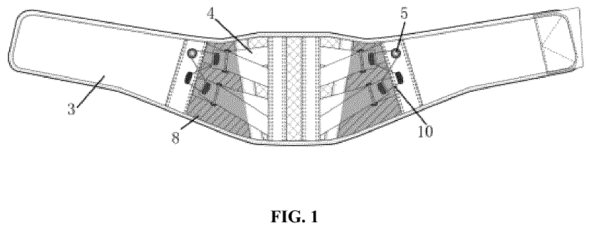

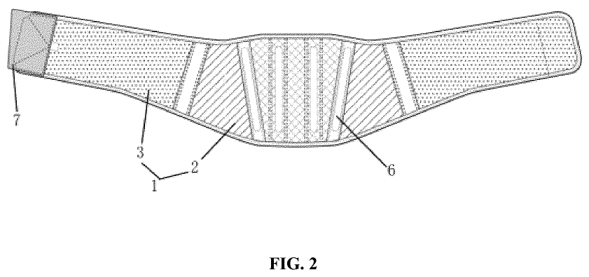

Referring to FIG. 1 and FIG. 2, in some embodiments of the present disclosure, a novelty of belt with knob for adjustable elasticity is provided, including a belt body 1. The belt body 1 includes a support part 2 and fixing belts 3 connected to both sides of the support part 2, the support part 2 is configured to be connected with a plurality of adjusting belts 4, and the adjusting belts 4 are arranged symmetrically along a center of the support part 2 to the both sides. Two fixing belts 3 are configured with adjusting knobs 5, the adjusting knobs 5 are configured with elastic strings 10, and the adjusting knobs 5 are connected with the adjusting belts 4 of corresponding sides by linkage of the elastic strings 10. Turning the adjusting knobs 5 can wind the elastic strings 10 into the adjusting knobs 5, the elastic strings 10 drive the adjusting belts 4 to pull to the both sides, which plays a tightening role, so as to achieve an adjustment effect. The elastic strings 10 are inherently elastic and does not break easily, and an elastic deformation of the elastic strings 10 can be adapted to actions of contraction and expansion of human muscles.

In some preferred embodiments of the present disclosure, the adjusting belts 4 located on the same side and any two adjacent adjusting belts 4 are in a group, and are sewn together in a “V” pattern, which can provide a more uniform force when shrinking and tightening.

Referring to FIG. 1, it shows a schematic diagram of a belt in accordance with some embodiments of the present disclosure. Herein any two adjacent adjusting belts 4 sewn together in the “V” pattern are provided with a first buckle on ends, and a second buckle is provided under the adjusting knobs 5, a elastic string 10 extends out from the adjusting knob 5 and then passes through the first buckle of one group of the adjust belts 4 (which is a group close to the adjusting knobs 5), follow passes through another second buckle, the first buckle of another group of the adjusting belts, and finally passes through the adjusting knob 5, the elastic string 10 is therefore wrapped in an “8” pattern.

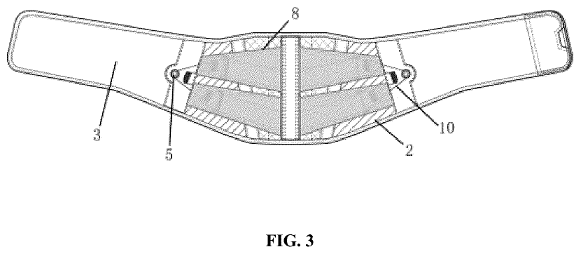

Referring to FIG. 3, it shows still another schematic diagram of a belt in accordance with some embodiments of the present disclosure. Herein any two adjacent adjusting belts 4 sewn together in the “V” shape are provided with a first buckle on ends, and a second buckle is provided under the adjusting knobs 5 near one side of the support part 2, a elastic string 10 extends from the adjusting knob 5 and then passes through the first buckle of one group of the adjust belts 4, follow passes through the second buckle, the first buckle of another group of the adjusting belts, and finally passes through the adjusting knob 5, the elastic string 10 is therefore wrapped in the “V” pattern.

In some preferred embodiments of the present disclosure, the adjusting knobs 5 are one-way adjusting knobs. The one-way adjusting knobs can prevent a sliding direction of the elastic strings, thus affecting an accuracy of the elasticity. The adjusting knobs 5 are provided with knobs, which can be pulled upward for a distance, and meanwhile, the knobs are not in a complete contact with the elastic strings, the elastic strings can be pulled outward to increase a length of the elastic strings adjusting the knobs. The knobs are pressed downward, at this time the knobs are in the complete contact with the elastic strings, the elastic strings cannot be pulled outward, but only wrap up to an inside of the adjusting knobs.

In some preferred embodiments of the present disclosure, the support part 2 is provided with a plurality of evenly spaced support strips 6 inside, and the support strips 6 can provide the support part 2 with excellent support force to better protect the waist.

In some preferred embodiments of the present disclosure, either of the fixing belts 3 is configured with Velcro 7, and the two fixing belts 3 are configured with loops adapted to the Velcro 7. Bonding of the Velcro 7 with the loops is only one of examples, and does not limit connection methods of the two fixing belts 3. For example, by means of snap fasteners, herein the loops are not only capable of bonding with the Velcro, but also an excellent tactile feeling can enhance the user's sense of experience.

In some preferred embodiments of the present disclosure, the fixing belts 3 also have gauzes 8 sewn to them, the gauzes 8 are located above the elastic strings, which is mainly used to cover the elastic strings 10, to prevent the elastic strings 10 from sleeving with other things, and the gauzes 8 also can cover the elastic strings 10, to make an appearance of the belt body 1 more beautiful. And there are openings between the gauzes 8 and the fixing belts 3 for the elastic strings 10 to pass through, such designs can ensure a proper adjustment of the elastic strings.

In some preferred embodiments of the present disclosure, a plurality of air holes is uniformly provided on an inner side of the fixing belts 3. The air holes fit with the loops, which can improve a breathability of the belt body 1 and thus enhance the user's sense of experience.

It is apparent to the person of ordinary skill in the art that the present disclosure is not limited to the details of the above-described exemplary embodiments, and that it is possible to realize this invention in other specific forms without departing from the spirit or essential features of the invention. Accordingly, in every point of view, the embodiments are to be regarded as exemplary and non-limiting, and the scope of the invention is limited by the appended claims and not by the foregoing description, and it is therefore intended that all variations falling within the meaning and scope of equivalent elements of the claims should be covered within the invention, any reference signs in the specification should not be regarded as limiting the claims involved.

What is claimed is:

1. A belt with knob for adjustable elasticity, comprising a belt body;
wherein the belt body comprises a support part and fixing belts connected to both sides of the support part,
the support part is configured to be connected with a plurality of adjusting belts, and the adjusting belts are arranged symmetrically along a center of the support part to the both sides; and
two fixing belts are configured with adjusting knobs, the adjusting knobs are configured with elastic strings, and the adjusting knobs are connected with the adjusting belts of corresponding sides by linkage of the elastic strings.

2. The belt with knob for adjustable elasticity according to claim 1, wherein the adjusting knobs are one-way adjusting knobs.

3. The belt with knob for adjustable elasticity according to claim 1, wherein the support part is configured with a plurality of evenly spaced support strips inside.

4. The belt with knob for adjustable elasticity according to claim 1, wherein either of the fixing belts is configured with Velcro, and the two fixing belts are configured with loops adapted to the Velcro.

5. The belt with knob for adjustable elasticity according to claim 1, wherein the fixing belts also have gauzes sewn to them, the gauzes are located above the elastic strings, and there are openings between the gauzes and the fixing belts for the elastic strings to pass through.

6. The belt with knob for adjustable elasticity according to claim 1, wherein a plurality of air holes is uniformly provided on an inner side of the fixing belts.
